(19) United States
(12) Patent Application Publication
(10) Pub. No.: US 2013/0331347 A1
(43) Pub. Date: Dec. 12, 2013

(54) Treatment of Clostridium Difficile Infection in Patients Undergoing Antibiotic Therapy

(71) Applicant: Optimer Pharmaceuticals, Inc., Jersey City, NJ (US)

(72) Inventors: Youe-Kong Shue, Taipei (TW); Sherwood Gorbach, San Diego, CA (US); Pamela Sears, San Diego, CA (US)

(73) Assignee: Optimer Pharmaceuticals, Inc., Jersey City, NJ (US)

(21) Appl. No.: 13/930,969

(22) Filed: Jun. 28, 2013

Related U.S. Application Data
(63) Continuation of application No. 13/698,958.
(60) Provisional application No. 61/345,915, filed on May 18, 2010.

(51) Int. Cl. A61K 31/7048

(52) U.S. Cl. CPC A61K 31/7048
USPC 514/28

(57) ABSTRACT

The present invention relates to methods of treating Clostridium difficile infection in a subject receiving antibiotic therapy for a different infection comprising administering to the subject an effective amount of the compounds described herein.

### FIELD OF THE INVENTION

The present invention relates to methods of treating Clostridium difficile infection (CDI) in a subject receiving antibiotic therapy for a different infection comprising administering to the subject an effective amount of the compounds described herein.

### BACKGROUND OF THE INVENTION

Clostridium difficile (C. difficile) is an anaerobic spore-forming bacterium that causes an infection of the bowel. Diarrhea is the most common symptom, but abdominal pain and fever may also occur. C. difficile is a major causative agent of colitis (inflammation of the colon) and diarrhea that may occur following antibiotic intake. This bacterium is primarily acquired in hospitals and chronic care facilities.

C. difficile-associated diarrhea (CDAD) is a disease characterized by severe and painful diarrhea. C. difficile is responsible for approximately 20% of the cases of antibiotic-associated diarrhea (AAD) and the majority of the cases of antibiotic-associated colitis (AAC). These diseases are typically caused by toxin-producing strains of C. difficile, S. aureus including methicillin-resistant S. aureus (MRSA) and Clostridium perfringens (C. perfringens). AAD represents a major economic burden to the healthcare system that is conservatively estimated at $3-6 billion per year in excess hospital costs in the U.S. alone.

Vancomycin-resistant enterococci (VRE), for which intestinal colonization provides a constant reservoir for infection, has also emerged as a major nosocomial pathogen associated with increased health care cost and mortality. VRE can appear as co-infection in patients infected with C. difficile, or more commonly cause infection in certain high risk patients such as haematology and oncology patients, patients in intensive care units and patients receiving solid organ transplants.

Methicillin-resistant Staphylococci, such as MRSA, are increasing in prevalence in both the hospital and community settings. Staphylococci are found on the skin and within the digestive and respiratory tracts but can infect open wounds and burns and can progress to serious systemic infection. The emergence of multi-drug resistant Staphylococci, especially, in the hospital where antibiotic use is frequent and selective pressure for drug-resistant organisms is high, has proven a challenge for treating these patients. The presence of MRSA on the skin of patients and health care workers promotes transmission of the multi-drug resistant organisms.

Similar diseases, including but not limited to clostridial enterocolitis, neonatal diarrhea, antibiotic-associated enterocolitis, sporadic enterocolitis, and nosocomial enterocolitis are also significant problems in some animal species.

There are currently two dominant therapies for CDAD: vancomycin and metronidazole. Vancomycin is not recommended for first-line treatment of CDAD mainly because it is the only antibiotic active against some serious life-threatening multi-drug resistant bacteria. Therefore, in an effort to minimize the emergence of vancomycin-resistant Enterococcus (VRE) or vancomycin-resistant S. aureus (VRSA), the medical community discourages the use of this drug except when absolutely necessary.

Metronidazole is recommended as initial therapy out of concern for the promotion and selection of vancomycin resistant gut flora, especially enterococci. Despite reports that the frequency of C. difficile resistance may be >6% in some countries, metronidazole remains nearly as effective as vancomycin, is considerably less expensive, and can be used either orally or intravenously. Metronidazole is associated with significant adverse effects including nausea, neuropathy, leukopenia, seizures, and a toxic reaction to alcohol. Furthermore, it is not safe for use in children or pregnant women.

Tiacumicins, specifically Tiacumicin B, show activity against a variety of bacterial pathogens and in particular against C. difficile (Antimicrob. Agents Chemother. 1991, 1108-1111). Because Tiacumicin B shows promising activity against C. difficile, it is expected to be useful in the treatment of bacterial infections, especially those of the gastrointestinal tract, in mammals. Examples of such treatments include but are not limited to treatment of colitis and treatment of irritable bowel syndrome.

Tiacumicin antibiotics are described in U.S. Pat. No. 4,918,174, J. Antibiotics 1987, 40:575-588, J. Antibiotics 1987, 40:567-574, J. Liquid Chromatography 1988, 11:191-201, Antimicrobial Agents and Chemotherapy 1991, 35:1108-1111, U.S. Pat. No. 5,583,115, and U.S. Pat. No. 5,767,096. Related compounds are the Lipiarmycin antibiotics (c.f., J. Chem. Soc. Perkin Trans. I, 1987, 1353-1359 and J. Antibiotics 1988, 41:308-315) and the Clostomicin antibiotics (J. Antibiotics 1986, 39:1407-1412).

Systemic infections (SI) requiring the administration of concomitant antibiotics (CAs) often complicate the treatment of CDI. In fact, traditionally, discontinuation of CA has been considered key in the management of CDI. An aspect of the present invention relates to the administration of a tiacumicin antibiotic, such as a compound of Formula I, to a subject for the treatment of CDI while the subject is receiving CAs for a different infection.

### SUMMARY OF THE INVENTION

The present invention relates to a method of treating Clostridium difficile infection (CDI) in a subject currently receiving antibiotic therapy for treatment of a different infection, comprising administering to the mammal an amount of a compound of Formula I below:

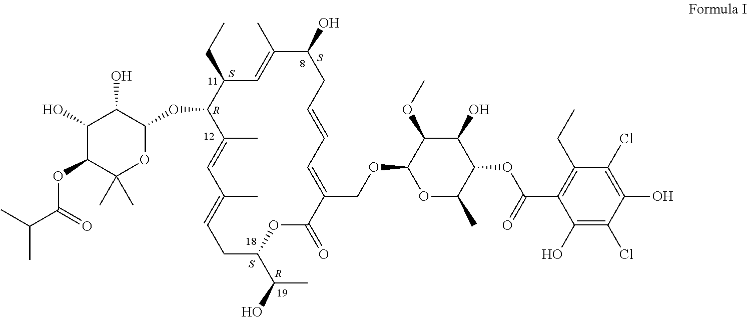

effective to treat the CDI, wherein the antibiotic therapy for treatment of a different infection does not involve administration of the compound of Formula I.

In an exemplary embodiment, the different infection is due to a bacterium, fungus or protozoan.

In an exemplary embodiment, the different infection is due to a gram-positive bacterium or a gram-negative bacterium.

In an exemplary embodiment, the different infection is due to a gram-negative bacterium.

In an exemplary embodiment, the antibiotic therapy for treatment of a different infection comprises administration of an antibiotic selected from the group consisting of aminoglycosides, ansamycins, carbacephems, carbapenems, cephalosporins, glycopeptides, lincosamides, macrolides, monobactams, penicillins, polypeptides, quinolones, rifamycins, sulfonamides and tetracyclines.

In an exemplary embodiment, the aminoglycoside antibiotic is selected from the group consisting of amikacin, gentamycin, kanamycin, neomycin, netilmicin, streptomycin, tobramycin and paromomycin.

In an exemplary embodiment, the ansamycin antibiotic is selected from the group consisting of geldanamycin and herbimycin.

In an exemplary embodiment, the carbapenem antibiotic is selected from the group consisting of ertapenem, doripenem, imipenem/cilastatin and meropenem.

In an exemplary embodiment, the cephalosporin antibiotic is selected from the group consisting of cefadroxil, cefazolin, cefalotin, cefalexin, cefaclor, cefamandole, cefoxitin, cefprozil, cefuroxime, cefiximie, cefdinir, cefditoren, cefoperazone, cefotaxime, cefpodoxime, ceftazidime, ceftibuten, ceftizoxime, ceftriaxone, cefepime and ceftobiprole.

In an exemplary embodiment, the glycopeptide antibiotic is selected from the group consisting of teicoplanin and vancomycin.

In an exemplary embodiment, the lincosamide antibiotic is selected from the group consisting of clindamycin and lincomycin.

In an exemplary embodiment, the macrolide antibiotic is selected from the group consisting of azithromycin, clarithromycin, dirithromycin, erythromycin, roxithromycin and telithromycin.

In an exemplary embodiment, the monobactam antibiotic is azactam.

In an exemplary embodiment, the penicillin antibiotic is selected from the group consisting of amoxicillin, ampicillin, azlocillin, carbenicillin, cloxacillin, dicloxacillin, flucloxacillin, mezlocillin, meticillin, nafcillin, oxacillin, penicillin, piperacillin and ticarcillin.

In an exemplary embodiment, the polypeptide antibiotic is selected from the group consisting of daptomycin, bacitracin, colistin and polymyxin B.

In an exemplary embodiment, the quinolone antibiotic is selected from the group consisting of ciprofloxacin, enoxacin, gatifloxacin, levofloxacin, lomefloxacin, moxifloxacin, norfloxacin, ofloxacin, trovafloxacin, grepafloxacin, sparfloxacin and temafloxacin.

In an exemplary embodiment, the rifamycin antibiotic is selected from the group consisting of rifamycin A, B, C, D, E, S and SV, rifaximin, rifampicin, rifabutin and rifapentine.

In an exemplary embodiment, the sulfonamide antibiotic is selected from the group consisting of mafenide, sulfonamidochrysoidine, sulfacetamide, sulfadizine, sulfamethizole, sulfanilimide, sulfasalazine, sulfisoxazole, trimethoprim and trimethoprim-sulfamethoxazole.

In an exemplary embodiment, the tetracycline antibiotic is selected from the group consisting of demeclocycline, doxycycline, minocycline, oxytetracycline and tetracycline.

In an exemplary embodiment, the different infection is selected from the group consisting of a respiratory infection, a mycoplasmal infection, Lyme disease, syphilis, gonorrhea, a chlamydial infection, malaria, pneumonia, an eye infection, a bladder infection, an urinary tract infection, otitis media, sinusitis, bronchitis, tonsillitis, pharynigis, rheumatic fever, uncomplicated skin and soft tissue infections, abscesses, conjunctivitis, keratitis, urethritis, cervicitis, osteomyelitis, bacterial prostatitis, salmonella and pseudo-membranous colitis.

In an exemplary embodiment, the different infection is selected from the group consisting of infections due to Clostridium perfringens, Staphylococcus spp., methicillin-resistant Staphylococcus, Streptococcus spp., Enterococcus spp., Haemophilus spp., Moraxella catarrhalis, Peptostreptococcus spp., Clostridium diptheriae, Actinobacillus haemolyticum; Mycoplasma pneumoniae, Legionella pneumophila, Corynebacterium minutissimum, Bartonella henselae, Treponema pallidum, Ureaplasma urealyticum, Neiserria gonorrhea, Helicobacter pylori, Borrelia recurrentis, Borrelia burgdorferi, Listeria spp.; Mycobacterium spp., Campylobacter jejuni, Cryptosporidium spp.; Bordetella pertussis, Bacteroides spp., E. coli, Serpulina hyodyisinteriae, Fusobacterium spp., Alcaligenes spp., Eubacterium spp., Peptostreptococcus spp., Porphyromonas spp. and Prevotella spp.

In an exemplary embodiment, the compound of Formula I is administered as a pharmaceutical composition.

In an exemplary embodiment, the pharmaceutical composition of Formula I further comprises butylated hydroxy toluene.

In an exemplary embodiment, the pharmaceutical composition of Formula I is administered orally.

In an exemplary embodiment, the antibiotic therapy for treatment of a different infection comprises administration of an antibiotic by an intramuscular, intraperitoneal, intranasal, oral, sublingual, intravaginal or rectal route.

In an exemplary embodiment, the antibiotic is administered as a pharmaceutical composition comprising an excipient.

In an exemplary embodiment, the compound of Formula I contains at least 93% of the R-stereoisomer.

In an exemplary embodiment, the subject is a mammal. In a particular embodiment, the mammal is a human.

### DETAILED DESCRIPTION OF THE INVENTION

The present invention relates to a method of treating CDI in a mammal currently receiving antibiotic therapy for treatment of a different infection, comprising administering to the mammal an amount of a compound of Formula I below:

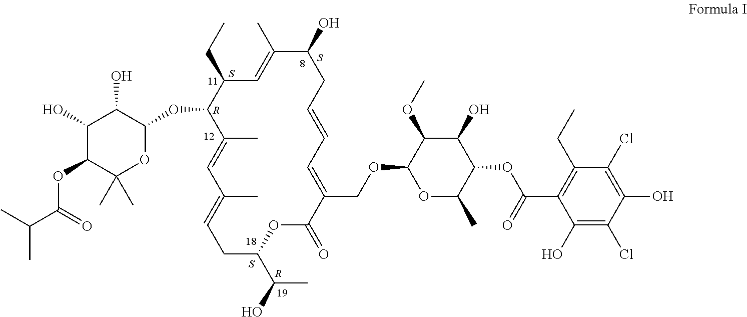

effective to treat the CDI, wherein the antibiotic therapy for treatment of a different infection does not involve administration of the compound of Formula I.

As used herein, “fidaxomicin” refers to the therapeutically active agent tested in the Examples described herein that comprises the compound of Formula I.

As used herein, the term “treatment” indicates a procedure which is designed ameliorate one or more causes, symptoms, or untoward effects of a bacterial infection in a subject. Likewise, the term “treat” is used to indicate performing a treatment. The treatment can, but need not, cure the subject, i.e., remove the cause(s), or remove entirely the symptom(s) and/or untoward effect(s) of the bacterial infection in the subject. Thus, a treatment may include treating a subject to inhibit the growth or proliferation of bacteria or protozoa, e.g., C. difficile, in the subject, or it may attenuate symptoms such as, but not limited to, diarrhea, fever, cramps, dehydration and peritonitis, or may include removing or decreasing the severity of the root cause of the bacterial infection in the subject. Treatment of a bacterial infection also includes treating after-arising symptoms that are related to the initial infection, such as diarrhea, fever, cramps, dehydration and peritonitis. As used herein, the term “subject” is used interchangeably with the term “patient,” and is used to mean an animal, in particular a mammal, and even more particularly a non-human or human primate.

A “bacterial infection” is used herein as it is used in the art, and the phrase is also used herein to include protozoal infections as well as disorders, conditions or symptoms associated with the bacterial infection and/or protozoal infections. In one embodiment, the bacterial infection is an infection of Clostridium difficile (C. difficile), Staphylococcus species, including but not limited to methicillin-resistant S. aureus (MRSA), Enterococcus species including but not limited to vancomycin-resistant Enterococci (VRE) or Clostridium perfringens (C. perfringens). The bacterial infection can be in any system, organ, tissue or area of the subject, such as but not limited to, gastrointestinal including upper and/or lower portions thereof, urinary, skin, ocular, auditory, blood, and respiratory to name a few.

In one embodiment, the bacterial infection is a first-time gastrointestinal (GI) infection of C. difficile, while in another embodiment, the bacterial infection is a recurring (GI) infection of C. difficile. As used herein, a recurring bacterial infection is an infection wherein the infection or the symptoms thereof occurs at an additional point in time, including more than once. The previous or initial infection or symptoms thereof may or may not have been treated prior to the reoccurrence of the infection or symptoms thereof. In one embodiment, the subject was not previously treated for the recurrent GI infection of C. difficile. In another embodiment, the subject was previously treated for the recurrent GI infection of C. difficile.

In a particular embodiment, the subject was not previously treated for the GI infection of C. difficile, while in another particular embodiment, the subject was previously treated. In these cases, the subject was treated with a composition or substance not including the compound of Formula I. Substances or compositions that may be used in these embodiments include any known antibiotic, including but not limited to, metronidazole, vancomycin, fusidic acid, rifaximin, bacitracin, tetracyclines, fluoroquinolones and/or teicoplanin. In other embodiments, the subject was previously treated for the GI infection of C. difficile and was treated with a composition or substance comprising compound of Formula I.

Other bacterial infections and disorders related to such infections include but are not limited to disorders associated with the use of antibiotics, chemotherapies, or antiviral therapies, including, but not limited to, colitis, for example, pseudo-membranous colitis, antibiotic associated diarrhea. More specifically, antibiotic-associated diarrhea caused by toxin producing strains of C. difficile, S. aureus including methicillin-resistant S. aureus, and C. perfringens. Others include antibiotic-associated colitis, pneumonia, otitis media, sinusitis, bronchitis, tonsillitis and mastoiditis related to infection by S. pneumoniae, Haemophilus influenzae, Moraxella catarrhalis, S. aureus, or Peptostreptococcus spp., pharynigis, rheumatic fever and glomerulonephritis related to infection by S. pyogenes, Groups C and G streptococci, C. diptheriae or Actinobacillus haemolyticum. Still others include respiratory tract infections related to infection by Mycoplasma pneumoniae, Legionella pneumophila, Streptococcus pneumoniae, Haemophilus influenzae, or Chlamydia pneumoniae, uncomplicated skin and soft tissue infections, abscesses and osteomyelitis, and puerperal fever related to infection by S. aureus, coagulase-positive Staphlococci (e.g., S. epidermis and S. hemolyticus), S. pyogenes, S. agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Neiserria gonorrhea. Other include toxin diseases related to infection by S. aureus (food poisoning and Toxic Shock Syndrome), or Groups A, B and C streptococci; ulcers related to infection by Helicobacter pylori, systemic febrile syndromes related to infection by Borrelia recurrentis; Lyme disease related to infection by Borrelia burgdorferi, conjunctivitis, keratitis, and dacrocystitis related to infection by Chlamydia trachomatis, Neisseria gonorrhoeae, S. aureus, S. pneumoniae, S. pyogenes, H. influenzae, or Listeria spp. Others include disseminated Mycobacterium avium complex (MAC) disease related to infection by Mycobacterium avium, or Mycobacterium intracellular; gastroenteritis related to infection by Campylobacter jejuni, intestinal protozoa related to infection by Cryptosporidium spp., odontogenic infection related to infection by viridans streptococci; persistent cough related to infection by Bordetella pertussis, gas gangrene related to infection by C. perfringens or Bacteroides spp., and atherosclerosis related to infection by H. pylori or Chlamydia pneumoniae. Other bacterial infections that may be treated, prevented or the likelihood of occurrence of which may be reduced in accord with the methods of the invention are referred to in Sanford, J. P., et al., The Sanford Guide To Antimicrobial Therapy, 40th Edition (Antimicrobial Therapy, Inc., 2010). Any of the bacterial infections or disorders or symptoms thereof may or may not be recurring.

Methods of treating or preventing a bacterial infection or a recurring infection described herein comprise administering a pharmaceutically effective amount of the compound of Formula I to a subject. As used herein, the term “administer” and “administering” are used to mean introducing the compound of Formula I into a subject. When administration is for the purpose of treatment, the substance is provided at, or after the onset of, a symptom of a bacterial infection. The therapeutic administration of this substance serves to attenuate any symptom, or prevent additional symptoms from arising. When administration is for the purposes of preventing or reducing the likelihood a bacterial infection or a recurrent (“prophylactic administration”), the substance is provided in advance of any visible or detectable symptom, such as after the symptoms of the initial infection. The prophylactic administration of the substance serves to attenuate subsequently arising symptoms or prevent or reduce the likelihood of the symptoms from arising altogether. Accordingly, the compound of Formula I may be used for the prevention of one disease or disorder and concurrently treating another (e.g., prevention of AAC, while treating urinary AAD.

The route of administration of the compound includes, but is not limited to, oral (such as a tablet, capsule or oral suspension), topical, transdermal, intranasal, vaginal, rectal, intraarterial, intramuscular, intraosseous, intraperitoneal, epidural and intrathecal.

Furthermore, the methods of treating or preventing a bacterial infection of the present invention also relate to co-administering one or more substances in addition to the compound of Formula I to the subject. The term “co-administer” indicates that each of at least two compounds are administered during a time frame wherein the respective periods of biological activity or effects overlap. Thus, the term includes sequential as well as coextensive administration of compounds. And similar to administering compounds, co-administration of more than one substance can be for therapeutic and/or prophylactic purposes. If more than one substance or compound is co-administered, the routes of administration of the two or more substances need not be the same. The scope of the invention is not limited by the identity of the substance which may be co-administered with the compound of Formula I. For example, the compound of Formula I may be co-administered with another pharmaceutically active substances, such as any known antibiotic. Alternatively, compositions comprising the compound of Formula I may be co-administered with fluids or other substances that are capable of alleviating, attenuating, preventing or removing symptoms in a subject suffering from, exhibiting the symptoms of, or at risk of suffering from a bacterial infection. Types of fluid that can be co-administered with the compound of Formula I should be specific to the circumstances surrounding the particular subject that is suffering from, exhibiting the symptoms of, or at risk of suffering from a bacterial infection. For example, fluids that may be co-administered with the compound of Formula I include but are not limited to, electrolytes and/or water, salt solutions, such as sodium chloride and sodium bicarbonate, as well as whole blood, plasma, serum, serum albumin and colloid solutions.

As used herein and unless otherwise indicated, the phrase “therapeutically effective amount” (or “pharmaceutically effective amount”) of the compound of Formula I or a pharmaceutically acceptable salt or prodrug thereof is measured by the therapeutic effectiveness of a compound of the invention, wherein at least one adverse effect of a disorder is ameliorated or alleviated. In one embodiment, the term “therapeutically effective amount” means an amount of the compound of Formula I that is sufficient to provide the desired local or systemic effect and performance at a reasonable benefit/risk ratio attending any medical treatment. The response to the therapeutically effective amount may be a cellular, organ or tissue-specific response, or system or systemic response. In one embodiment, the phrase “therapeutically effective amount” of a composition of the invention is measured by the therapeutic effectiveness of a compound of the invention to alleviate at least one symptom associated with bacterial or protozoal infections. Examples of therapeutically effective amounts include, but are not limited to those in the Examples section herein.

As used herein and unless otherwise indicated, the term “binders” refers to agents used to impart cohesive qualities to the powdered material. Binders, or “granulators” as they are sometimes known, impart cohesiveness to the tablet formulation, which insures the tablet remaining intact after compression, as well as improving the free-flowing qualities by the formulation of granules of desired hardness and size. Materials commonly used as binders include starch, gelatin, sugars, such as sucrose, glucose, dextrose, molasses, and lactose, natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, larch arabogalactan and the like.

As used herein and unless otherwise indicated, the term “carrier” refers to a diluent, adjuvant, excipient, or vehicle with which a composition is administered. Such pharmaceutical carriers can be sterile liquids, such as water and oils, including those of petroleum, animal, vegetable or synthetic origin, such as peanut oil, soybean oil, mineral oil, sesame oil and the like.

As used herein and unless otherwise indicated, the term “compounds of the invention” means, collectively, a compound of Formula I and/or pharmaceutically acceptable salts, solvates, hydrates, amorphous forms and polymorphs thereof. The compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain one or more chiral centers and/or double bonds and may therefore exist as stereoisomers, such as double-bond isomers (i.e., geometric isomers), enantiomers, or diastereomers. According to the invention, the chemical structures depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures, and solvates and/or hydrates thereof. Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods.

In one embodiment, the pharmaceutical compositions used in the methods of the present invention comprise the compound of Formula I that is substantially stereomerically pure. In specific embodiments, the pharmaceutical compositions comprise the compound of Formula I that is at least about 75% pure, 80%, 85%, 90%, 91%, 92%, 93%, 94%, 95%, 96%, 97%, 98% or 99% pure, i.e., free from other stereoisomers, diastereoisomers, enantiomers, etc.

As used herein and unless otherwise indicated, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein and unless otherwise indicated, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

The term “MIC” or “minimum inhibitory concentration” refers to the lowest concentration of an antibiotic that is needed to inhibit growth of a bacterial isolate in vitro. A common method for determining the MIC of an antibiotic is to prepare several tubes containing serial dilutions of the antibiotic, that are then inoculated with the bacterial isolate of interest. The MIC of an antibiotic can be determined from the tube with the lowest concentration that shows no turbidity (no growth). The term “MIC50” refers to the lowest concentration of antibiotic required to inhibit the growth of 50% of the bacterial strains tested within a given bacterial species. The term “MIC90” refers to the lowest concentration of antibiotic required to inhibit the growth of 90% of the bacterial strains tested within a given bacterial species.

As used herein and unless otherwise indicated, the term “mixture of tiacumicins” refers to a composition containing at least one macrolide compound from the family of compounds known tiacumicins. In another embodiment, the term “mixture of tiacumicins” includes a mixture containing at least one member of the compounds known tiacumicins and the compound of Formula I, wherein the compound of Formula I is present in an amount of at least about 50%, 60%, 70%, 80%, 90%, 95%, 99%, 99.9%, or 99.99% by weight. In particular, the term “mixture of tiacumicins” refers to a compositions comprising the compound of Formula I, wherein the compound of Formula I has a relative retention time (“RTT”) ratio of 1.0, and further comprising at least one of compounds 101-112 in PCT Application No. PCT/US2008/000735.

As used herein, and unless otherwise indicated, the terms “optically pure,” “stereomerically pure,” and “substantially stereomerically pure” are used interchangeably and mean one stereoisomer of a compound or a composition that comprises one stereoisomer of a compound and is substantially free of other stereoisomer(s) of that compound. For example, a stereomerically pure compound or composition of a compound having one chiral center will be substantially free of the opposite enantiomer of the compound. A stereomerically pure compound or composition of a compound having two chiral centers will be substantially free of other diastereomers of the compound. A typical stereomerically pure compound comprises at least about 80% by weight of one stereoisomer of the compound, i.e., free from other stereoisomers, diastereoisomers, enantiomers, etc., and about 20% or less by weight of other stereoisomers of the compound, more specifically at least about 90% by weight of one stereoisomer of the compound and about 10% or less by weight of the other stereoisomers of the compound, even more specifically, at least about 95% by weight of one stereoisomer of the compound and about 5% or less by weight of the other stereoisomers of the compound, and more specifically, at least about 97% by weight of one stereoisomer of the compound and about 3% or less by weight of the other stereoisomers of the compound.

As used herein and unless otherwise indicated, “pharmaceutically acceptable” refers to materials and compositions that are physiologically tolerable and do not typically produce an allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein and unless otherwise indicated, the term “pharmaceutically acceptable hydrate” means the compound of Formula I that further includes a stoichiometric or non-stoichiometric amount of water bound by non-covalent intermolecular forces.

As used herein and unless otherwise indicated, the term “pharmaceutically acceptable polymorph” refers to the compound of Formula I that exists in several distinct forms (e.g., crystalline, amorphous), the invention encompasses all of these forms. In another embodiment, a pharmaceutically acceptable polymorph of a Compound of Formula I exhibits a representative powder diffraction pattern comprising at least peaks at the following diffraction angles 2Θ of 7.7°, 15.0°, and 18.8°±0.04, or ±0.1, or ±0.15, or ±0.2, as shown in FIG. 1. In another embodiment, a pharmaceutically acceptable polymorph of a Compound of Formula I exhibits a representative powder diffraction pattern comprising at least peaks at the following diffraction angles 2Θ of 7.6°, 15.4°, and 18.8°±0.04, or ±0.1, or ±0.15, or ±0.2, as shown in FIG. 2.

Methods of preparing and characterizing select embodiments of pharmaceutically acceptable polymorphs are found in International Application No. PCT/US2008/000735.

As used herein and unless otherwise indicated, the term “pharmaceutically acceptable prodrug” means a derivative of a modified polymorph of a compound of Formula I that can hydrolyze, oxidize, or otherwise react under biological conditions (in vitro or in vivo) to provide the compound of Formula I. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery 172, 178, 949, 982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound of Formula I but can confer upon that compound advantageous properties in vivo, such as but not limited to uptake, duration of action, or onset of action, or 2) is biologically inactive but is converted in vivo to the biologically active compound. Examples of biohydrolyzable esters include, but are not limited to, lower alkyl esters, lower acyloxyalkyl esters (such as acetoxyl methyl, acetoxyethyl, am inocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

The phrase “pharmaceutically acceptable salt(s),” as used herein includes but is not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds, included in the present compositions, which are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

In some embodiments, the methods of the invention encompass administering pharmaceutical compositions comprising a first polymorph of the compound of Formula I, a second polymorph of the compound of Formula I, other polymorphic forms, amorphous form or mixtures thereof of a mixture of tiacumicins with varying amounts of the compound of Formula I. Certain embodiments of the methods of the present invention may also comprise administering pharmaceutical compositions that are mixtures of tiacumicins for use in treating CDAD as well as AAD and AAC. In one specific embodiment, the mixture of tiacumicins contains from about 76% to about 100% of the compound of Formula I.

The compound of Formula I is useful in veterinary and human medicine for the treatment or prevention of bacterial and protozoal infections. In some embodiments, the subject has an infection but does not exhibit or manifest any physiological symptoms associated with an infection.

The present compositions, which comprise one or more crystalline polymorph or amorphous form of the compound of Formula I or the compound of Formula I within a mixture of tiacumicins may be administered by any convenient route, for example, peroral administration, parenteral administration, by infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a composition of the invention. In certain embodiments, more than one compound of Formula I and a mixture of tiacumicins is administered to a patient. Methods of administration include but are not limited to intradermal, intramuscular, intraperitoneal, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner, and will depend in part upon the site of the medical condition. In most instances, administration will result in the release of the crystalline polymorph or amorphous form of the compound of Formula I into the bloodstream.

In specific embodiments, it may be desirable to administer one or more crystalline polymorph or amorphous form of the compound of Formula I locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the a crystalline polymorph or amorphous form of the compound of Formula I can be delivered in a vesicle, in particular a liposome (see Langer, 1990, Science 249:1527-1533; Treat et al., in Liposomes in The Therapy of Infectious Disease and Cancer, Lopez-Berestein and Fidler (eds.), Liss, N.Y., pp. 353-365 (1989); Lopez-Berestein, ibid., pp. 317-327; see generally ibid.).

In yet another embodiment, the compositions of the invention can be delivered in a controlled release system. In one embodiment, a pump may be used (see Langer, supra; Sefton, 1987, CRC Crit. Ref Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the compound of Formula I, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions will contain a therapeutically effective amount of a crystalline polymorph or amorphous form of the compound of Formula I, optionally more than one crystalline polymorph or amorphous form of the compound of Formula I, for example in purified form, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which the compound of Formula I is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the invention and pharmaceutically acceptable vehicles are preferably sterile. Water is an example of a vehicle of the compounds of the invention. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's The Science and Practice of Pharmacy,” Berringer, P. et al. (Eds) Lippincott Williams & Wilkins (21^{st} Ed. 2006). The pharmaceutical compositions may contain preserving agents, solubilising agents, stabilising agents, wetting agents, emulsifiers, sweeteners, colorants, odorants, salts, buffers, coating agents or antioxidants, such as but not limited to butylated hydroxytoluene (BHT). They may also contain therapeutically active agents in addition to the substance of the present invention.

In one embodiment, the compositions of the invention are administered orally. Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered crystalline polymorph or amorphous form of the compound of Formula I. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a crystalline polymorph or amorphous form of the compound of Formula I that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. Suitable dosage ranges for oral administration, however, are generally from about 0.001 milligram to 1000 milligrams of the compound of Formula I per kilogram body weight. In one embodiment, the oral dose is about 0.01 milligram to about 500 milligrams per kilogram body weight, or from about 0.1 milligram to about 100 milligrams per kilogram body weight, or from about 0.5 milligram to about 50 milligrams per kilogram body weight. In a specific embodiment, the oral dose is from about 1 milligram to about 10 milligrams per kilogram body weight. In a more specific embodiment, the oral dose is about 1 milligram of a crystalline polymorph or amorphous form of the compound of Formula I per kilogram body weight. The dosage amounts described herein refer to total amounts administered; that is, if more than one compound is administered, the preferred dosages correspond to the total amount of the compounds of the invention administered. The oral compositions described herein may contain from about 10% to about 95% active ingredient by weight, and the oral compositions may be dosed 1, 2, 3, 4, 5 or more times daily.

Suitable dosage ranges for intranasal administration are generally from about 0.01 pg/kg body weight to about 1 mg/kg body weight of the compound of Formula I. Suppositories generally contain from about 0.01 milligram to about 50 milligrams of the compound of Formula I per kilogram body weight and comprise active ingredient in the range of from about 0.5% to about 10% by weight. Recommended dosages for intradermal, intramuscular, intraperitoneal, epidural, sublingual, intracerebral, intravaginal, transdermal administration or administration by inhalation are in the range of from about 0.001 milligram to about 1000 milligrams per kilogram of body weight of the compound of Formula I. Suitable doses of the compounds of the invention for topical administration are in the range of from about 0.001 milligram to about 1 milligram of the compound of Formula I, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more crystalline polymorph or amorphous form of the compound of Formula I. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one crystalline polymorph or amorphous form of the compound of Formula I.

Methods of manufacturing the compound of Formula I, including select polymorphs thereof are disclosed in U.S. Pat. No. 7,378,508.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1

### Production of Compound of Formula I

The compound of Formula I can be produced by fermentation. Cultivation with a mutant form derived from Dactylosporangium aurantiacum subspecies hamdenensis AB 718C-41 NRRL 18085 for the production was carried out in a medium containing carbon sources, inorganic salts and other organic ingredients with one or more absorbents under proper aeration conditions and mixing in a sterile environment. The production method is disclosed in U.S. Pat. No. 7,507,564.

The nutrient medium comprises from about 0.5 to about 15% of the adsorbent by weight. In one embodiment, the absorbent is an adsorbent substance, such as a resin. Examples of absorbent substances include but are not limited to Amberlite®, XAD 16, XAD 16HP, XAD2, XAD7HP, XADI 180, XAD 1600, IRC50, or Duolite® XAD761. The nutrient medium can comprise the following combination based on weight: from about 0.2% to about 10% of glucose, from about 0.02% to about 0.5% of K₂HPO₄, from about 0.02% to about 0.5% of MgSO₄.7H₂O, from about 0.01% to about 0.3% of KCl, from about 0.1% to about 2% of CaCO₃, from about 0.05% to about 2% of casamino acid, from about 0.05% to about 2% of yeast extract, and from about 0.5% to about 15% of XAD-16 resin. The culturing step was conducted at a temperature from about 25° C. to about 35° C. and at a pH from about 6.0 to about 8.0.

Upon completion of fermentation, the solid mass (including the adsorbent resin) was separated from the broth. The products were extracted with organic solvents such as, for example, ethyl acetate then concentrated under reduced pressure.

### Example 2

### Purification of Compound of Formula I

After the fermentation in Example 1, the crude material was purified by HPLC. The collected fractions containing about 90-99% of compound of Formula I were combined. The solid was crystallized to the desired crystalline form to produce the pharmaceutical composition (fidaxomicin). HPLC analysis showed fidaxomicin to contain about >93% of compound of Formula I as a major component and a mixture of tiacumicins as the minor component.

### Example 3

### Administration of Concomitant Antibiotics

Administration of concomitant antibiotics (CAs) to subjects during the 10 days of study drug administration (PO Fidaxomicin (FDX 200 mg BID)) vs. (PO Vancomycin (Vanc 125 mg QID)) through 4-wk follow-up period was reviewed. End points were the effect of CAs on clinical cure, CDI recurrence during 4-wk follow-up, and global cure (clinical cure with no recurrence).

Results: Per protocol (mITT results did not differ in any outcome).

| | Clinical Cure | | Recurrence | | Global Cure | |
|---|---|---|---|---|---|---|
| | No CA | CA | No CA | CA | No CA | CA |
| Combined | 93% | 80% | 15% | 33% | 76% | 59% |
| Vanc/ | (409/438) | (89/110) | (48/ | (33/ | (331/438) | (65/110) |
| FDX | | | 331^{†}) | 101^{†}) | | |
| p-value*¹ | p < 0.001 | | p < 0.001 | | p = 0.001 | |
| Vanc | 94% | 77% | 18% | 40% | 72% | 50% |
| (PP) | (205/219) | (49/64) | (30/164) | (23/57) | (158/219) | (32/64) |
| FDX | 93% | 87% | 11% | 23% | 79% | 72% |
| (PP) | (204/219) | (40/46) | (18/167) | (10/44) | (173/219) | (33/46) |
| p- | p = | p = | p = | p = | p = | p = |
| value*² | 0.848 | 0.171 | 0.052 | 0.061 | 0.095 | 0.022 |

*p-value calculated using a 2-sided normal approximation Z-test for 2 proportions.
^{†}116 subjects failed therapy or were lost to follow-up.
¹No CA vs CA.
²FDX vs Vanc.

Conclusion: CAs given during CDI treatment were associated with higher recurrences of CDI indicating an adverse outcome. In those who received CAs, CDI recurrences were lower and global cures were significantly improved in those treated with FDX compared to Vanc. FDX may be a more effective therapy for CDI in patients requiring CAs.

Although the present invention has been described in detail with reference to examples above, it is understood that various modifications can be made without departing from the spirit of the invention. Accordingly, the invention is limited only by the following claims. All patents, published patent applications, and other published references cited herein are incorporated by reference in their entirety.

1. A method of treating CDI in a mammal currently receiving antibiotic therapy for treatment of a different infection, comprising administering to the mammal an amount of a compound of Formula I 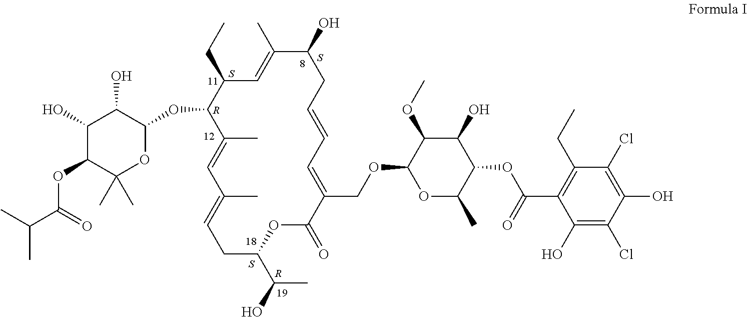
effective to treat the CDI, wherein the antibiotic therapy for treatment of a different infection does not involve administration of the compound of Formula I.

2. The method of claim 1 wherein the different infection is due to a bacterium, fungus or protozoan.

3. The method of claim 1 wherein the different infection is due to a gram-positive bacterium.

4. The method of claim 1 wherein the different infection is due to a gram-negative bacterium.

5. The method of claim 1 wherein the antibiotic therapy for treatment of a different infection comprises administration of an antibiotic selected from the group consisting of aminoglycosides, ansamycins, carbacephems, carbapenems, cephalosporins, glycopeptides, lincosamides, macrolides, monobactams, penicillins, polypeptides, quinolones, rifamycins, sulfonamides and tetracyclines.

6. The method of claim 5 wherein the aminoglycoside antibiotic is selected from the group consisting of amikacin, gentamycin, kanamycin, neomycin, netilmicin, streptomycin, tobramycin and paromomycin.

7. The method of claim 5 wherein the ansamycin antibiotic is selected from the group consisting of geldanamycin and herbimycin.

8. The method of claim 5 wherein the carbapenem antibiotic is selected from the group consisting of ertapenem, doripenem, imipenem/cilastatin and meropenem.

9. The method of claim 5 wherein the cephalosporin antibiotic is selected from the group consisting of cefadroxil, cefazolin, cefalotin, cefalexin, cefaclor, cefamandole, cefoxitin, cefprozil, cefuroxime, cefiximie, cefdinir, cefditoren, cefoperazone, cefotaxime, cefpodoxime, ceftazidime, ceftibuten, ceftizoxime, ceftriaxone, cefepime and ceftobiprole.

10. The method of claim 5 wherein the glycopeptide antibiotic is selected from the group consisting of teicoplanin and vancomycin.

11. The method of claim 5 wherein the macrolide antibiotic is selected from the group consisting of azithromycin, clarithromycin, dirithromycin, erythromycin, roxithromycin, troleandomycin, telithromycin and spectinomycin.

12. The method of claim 5 wherein the monobactam antibiotic is azactam.

13. The method of claim 5 wherein the penicillin antibiotic is selected from the group consisting of amoxicillin, ampicillin, azlocillin, carbenicillin, cloxacillin, dicloxacillin, flucloxacillin, mezlocillin, meticillin, nafcillin, oxacillin, penicillin, piperacillin and ticarcillin.

14. The method of claim 5 wherein the polypeptide antibiotic is selected from the group consisting of bacitracin, colistin and polymyxin B.

15. The method of claim 5 wherein the quinolone antibiotic is selected from the group consisting of ciprofloxacin, enoxacin, gatifloxacin, levofloxacin, lomefloxacin, moxifloxacin, norfloxacin, ofloxacin, trovafloxacin, grepafloxacin, sparfloxacin and temafloxacin.

16. The method of claim 5 wherein the sulfonamide antibiotic is selected from the group consisting of mafenide, sulfonamidochrysoidine, sulfacetamide, sulfadizine, sulfamethizole, sulfanilimide, sulfasalazine, sulfisoxazole, trimethoprim and trimethoprim-sulfamethoxazole.

17. The method of claim 5 wherein the tetracycline antibiotic is selected from the group consisting of demeclocycline, doxycycline, minocycline, oxytetracycline and tetracycline.

18. The method of claim 5 wherein the the lincosamide antibiotic is selected from the group consisting of clindamycin and lincomycin.

19. The method of claim 5 wherein the rifamycin antibiotic is selected from the group consisting of rifamycin A, B, C, D, E, S and SV, rifaximin, rifampicin, rifabutin and rifapentine.

20. The method of claim 1 wherein the different infection is selected from the group consisting of a respiratory infection, a mycoplasmal infection, Lyme disease, syphilis, gonorrhea, a chlamydial infection, malaria, pneumonia, an eye infection, a bladder infection, an urinary tract infection, otitis media, sinusitis, bronchitis, tonsillitis, pharynigis, rheumatic fever, uncomplicated skin and soft tissue infections, abscesses, conjunctivitis, keratitis, urethritis, cervicitis, osteomyelitis, bacterial prostatitis, salmonella and pseudo-membranous colitis.

21. The method of 1 claim wherein the different infection is selected from the group consisting of infections due to Clostridium perfringens, Streptococcus spp., Staphylococcus spp., methicillin-resistant Staphylococcus, Enterococcus spp., Haemophilus spp., Moraxella catarrhalis, Peptostreptococcus spp., Clostridium diptheriae, Actinobacillus haemolyticum; Mycoplasma pneumoniae, Legionella pneumophila, Corynebacterium minutissimum, Bartonella henselae, Treponema pallidum, Ureaplasma urealyticum, Neiserria gonorrhea, Helicobacter pylori, Borrelia recurrentis, Borrelia burgdorferi, Listeria spp.; Mycobacterium spp., Campylobacter jejuni, Cryptosporidium spp.; Bordetella pertussis, Bacteroides spp., E. coli, Serpulina hyodyisinteriae, Fusobacterium spp., Alcaligenes spp., Eubacterium spp., Peptostreptococcus spp., Porphyromonas spp. and Prevotella spp.

22. The method of claim 1 wherein the compound of Formula I is administered as a pharmaceutical composition.

23. The method of claim 20 wherein the pharmaceutical composition of Formula I further comprises butylated hydroxy toluene.

24. The method of claim 22 wherein the pharmaceutical composition of Formula I is administered orally.

25. The method of claim 1 wherein the antibiotic therapy for treatment of a different infection comprises administration of an antibiotic by a intramuscular, intraperitoneal, intranasal, oral, sublingual, intravaginal or rectal route.

26. The method of claim 5 wherein the antibiotic is administered as a pharmaceutical composition comprising an excipient.

27. The method of claim 1 wherein the compound of Formula I contains at least 93% of the R-stereoisomer.

28. The method of claim 1 wherein the mammal is a human.
